(19) United States
(12) Patent Application Publication
(10) Pub. No.: US 2019/0000417 A1
(43) Pub. Date: Jan. 3, 2019

(54) ENDOSCOPE

(71) Applicant: OLYMPUS CORPORATION, Tokyo (JP)

(72) Inventor: Yasuhiro IIJIMA, Tokyo (JP)

(73) Assignee: OLYMPUS CORPORATION, Tokyo (JP)

(21) Appl. No.: 16/125,936

(22) Filed: Sep. 10, 2018

Related U.S. Application Data
(63) Continuation of application No. PCT/JP2016/085635, filed on Nov. 30, 2016.

(30) Foreign Application Priority Data
Mar. 14, 2016 (JP) 2016-049594

(51) Int. Cl. A61B 8/00; A61B 1/00; A61B 1/018; A61B 8/12

(52) U.S. Cl. CPC A61B 8/445; A61B 1/00018; A61B 8/4483; A61B 8/12; A61B 1/018

(57) ABSTRACT

An endoscope includes: an insertion portion configured to be inserted into a subject; an image sensor configured to acquire an image of the subject; a distal end constituting portion that includes a mounting portion having a hole shape and used for mounting the image sensor, and two communication portions for allowing the mounting portion to communicate with outside of the distal end constituting portion; a signal cable that includes two signal line groups, the signal line groups including a plurality of signal lines configured to transmit signals acquired by the image sensor; a channel that is inserted into a space formed by the two signal line groups of the signal cable, and that allows an elongated member to be inserted in the channel; and a tubular portion that has a tubular shape and allows the signal cable and the channel to be inserted in the tubular portion.

### CROSS-REFERENCE TO RELATED APPLICATION

This application is a continuation of PCT International Application No. PCT/JP2016/085635 filed on Nov. 30, 2016 which claims the benefit of priority from Japanese Patent Application No. 2016-049594, filed on Mar. 14, 2016, the entire contents of which are incorporated herein by reference.

### BACKGROUND

### 1. Technical Field

The present disclosure relates to an endoscope.

### 2. Related Art

In the related art, a rigid or flexible endoscope is used at the time of observing organs of a subject, such as a patient, or materials. For example, an operator, such as a doctor, uses an endoscope, in which an ultrasound transducer that transmits and receives ultrasound waves is provided at a distal end of an insertion portion, and observes an observation target on the basis of information that relates to characteristics of the observation target and that is generated based on ultrasound echoes received from the ultrasound transducer.

The ultrasound transducer includes a plurality of piezoelectric elements, each of which converts an electrical pulse signal to an ultrasound pulse (acoustic pulse), applies the ultrasound pulse to the observation target, converts an ultrasound echo reflected by the observation target to an electrical echo, and outputs the electrical echo. Each of the piezoelectric elements is electrically connected to an ultrasound observation apparatus via a cable that includes a plurality of signal lines.

Meanwhile, there is a demand to reduce a diameter of the insertion portion of the endoscope. As a technology for reducing the diameter of the insertion portion, a technology for dividing some of the signal lines in the cable into a plurality of bundles to thereby avoid interference with an internal object has been known (for example, see JP 2005-342129 A).

### SUMMARY

In some embodiments, an endoscope includes: an insertion portion configured to be inserted into a subject; an image sensor configured to acquire an image of the subject; a distal end constituting portion that is provided at a distal end of the insertion portion, and that includes a mounting portion having a hole shape and used for mounting the image sensor, and two communication portions for allowing the mounting portion to communicate with outside of the distal end constituting portion; a signal cable that includes two signal line groups, the signal line groups having one ends connected to the image sensor, extending from the distal end constituting portion via the communication portions, and including a plurality of signal lines configured to transmit signals acquired by the image sensor; a channel that has a cylindrical shape, that is provided inside the insertion portion, that is inserted into a space formed by the two signal line groups of the signal cable, and that allows an elongated member to be inserted in the channel; and a tubular portion that has a tubular shape and allows the signal cable and the channel to be inserted in the tubular portion.

In some embodiments, an endoscope includes: a tubular portion that has a tubular shape; a channel that is inserted into the tubular portion and arranged to be inclined with respect to an axial direction of the tubular portion; a signal cable including two signal line groups by which a space is formed, the space allowing the channel to be inserted in the space; an ultrasound transducer connected to a distal end of the signal cable and configured to acquire information on a subject; a distal end constituting portion that is provided at a distal end of the tubular portion, and that includes a mounting portion on which the ultrasound transducer is mounted, a holding hole capable of holding an insulating pipe in which the signal cable is insertable, and a communication hole communicating with the channel; and an insulating tube configured to cover a part of each of the two signal line groups of the signal cable.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a perspective view schematically illustrating a rigid endoscope system according to a first embodiment of the present disclosure;

FIG. 2 is a perspective view schematically illustrating a configuration in a case where an optical viewing tube is mounted on a rigid endoscope body of the rigid endoscope system according to the first embodiment of the present disclosure;

FIG. 3 is a cross-sectional view schematically illustrating a configuration of a main part of the rigid endoscope body of the rigid endoscope system according to the first embodiment of the present disclosure;

FIG. 4 is a cross-sectional view schematically illustrating a configuration of a distal end of the rigid endoscope body of the rigid endoscope system according to the first embodiment of the present disclosure;

FIG. 5A is a cross-sectional view of the rigid endoscope body corresponding to line A-A illustrated in FIG. 4;

FIG. 5B is a cross-sectional view of the rigid endoscope body corresponding to line B-B illustrated in FIG. 4;

FIG. 5C is a cross-sectional view of the rigid endoscope body corresponding to line C-C illustrated in FIG. 4;

FIG. 5D is a cross-sectional view of the rigid endoscope body corresponding to line D-D illustrated in FIG. 4;

FIG. 5E is a cross-sectional view of the rigid endoscope body corresponding to line E-E illustrated in FIG. 4;

FIGS. 6A and 6B is a diagram for explaining a diameter of an insertion portion according to the first embodiment of the present disclosure and a diameter of a conventional insertion portion; and

FIG. 7 is a cross-sectional view schematically illustrating a configuration of a main part of a rigid endoscope body of a rigid endoscope system according to a second embodiment of the present disclosure.

### DETAILED DESCRIPTION

Modes (hereinafter, referred to as “embodiments”) for carrying out the present disclosure will be described below with reference to the drawings. The present disclosure is not limited by the embodiments below. Further, in the description of the drawings, the same components are denoted by the same reference signs.

### First Embodiment

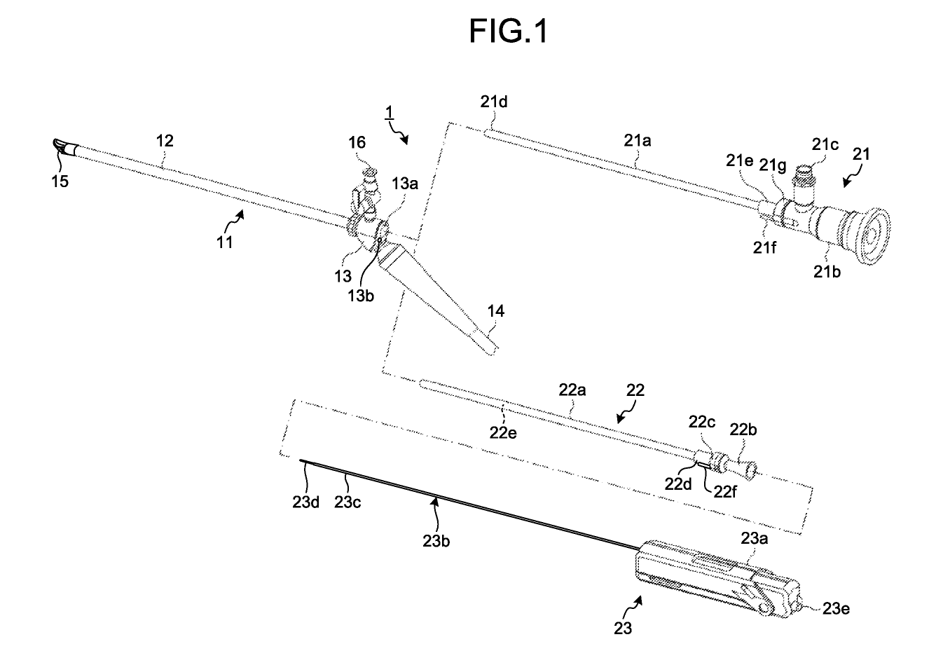

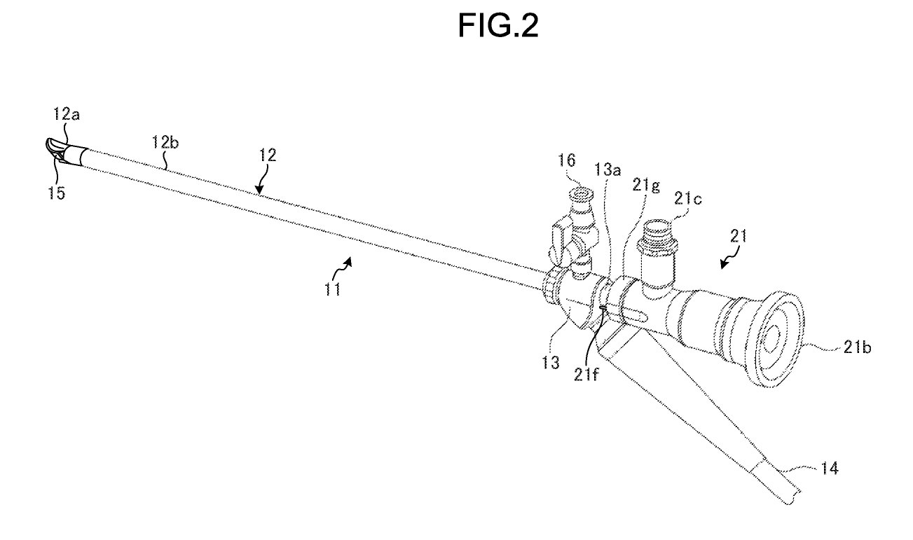

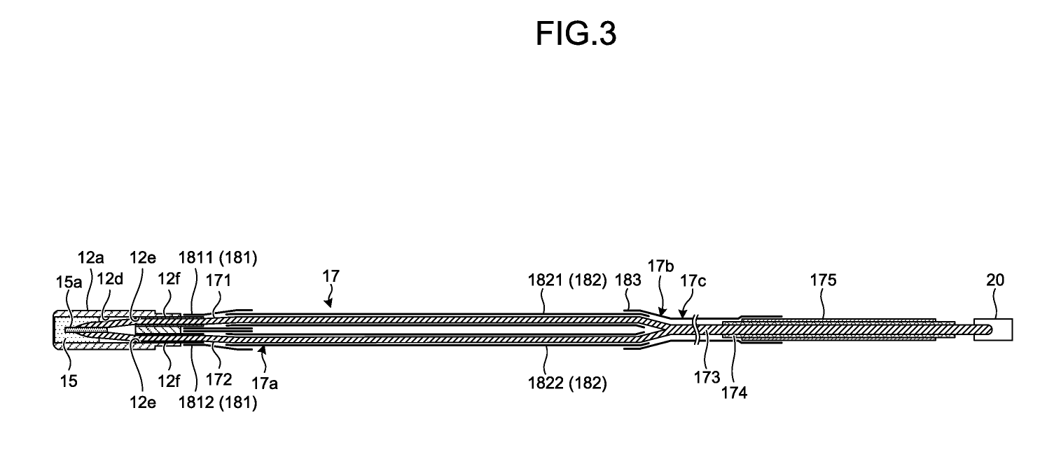

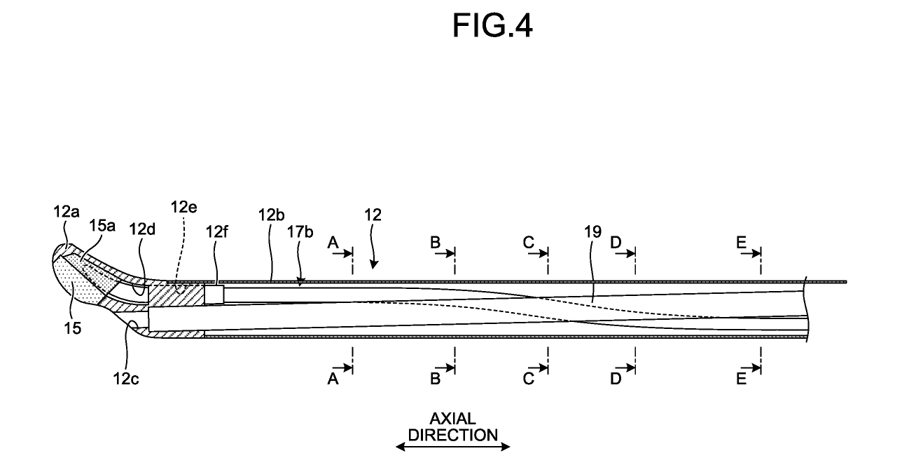

FIG. 1 is a perspective view schematically illustrating a rigid endoscope system according to a first embodiment of the present disclosure. FIG. 2 is a perspective view schematically illustrating a configuration in a case where an optical viewing tube is mounted on a rigid endoscope body of the rigid endoscope system according to the first embodiment of the present disclosure. FIG. 3 is a cross-sectional view schematically illustrating a configuration of a main part of the rigid endoscope body of the rigid endoscope system according to the first embodiment of the present disclosure, and is the cross-sectional view illustrating a configuration in a case where the rigid endoscope body is stretched linearly. FIG. 4 is a cross-sectional view schematically illustrating a configuration of a distal end of the rigid endoscope body of the rigid endoscope system according to the first embodiment of the present disclosure.

A rigid endoscope system 1 is a system that performs ultrasound diagnosis inside a subject, such as a human, using an ultrasound endoscope, and is used at the time of transurethral sampling of a biopsy tissue of the prostate, for example. The rigid endoscope system 1 includes a rigid endoscope body 11, an optical viewing tube 21 as an imaging device, a treatment tool guide 22, and a treatment tool device 23.

The rigid endoscope body 11 includes a first insertion portion 12 that is inserted into a lumen (for example, a urethra) of the subject, a grip portion 13 that is provided on a front side of the first insertion portion 12, and a universal cord 14 that extends from a side of the grip portion 13 opposite to a side at which the first insertion portion 12 is connected. FIG. 2 illustrates a configuration in a case where the optical viewing tube 21 is mounted on the rigid endoscope body 11 as one example of use modes of the rigid endoscope system 1.

The first insertion portion 12 is rigid and extends linearly. A signal cable 17 extending from the universal cord 14 is inserted through an inner lower side of the first insertion portion 12 along an axial direction. The first insertion portion 12 includes a distal end constituting portion 12a, which is provided at a distal end of the first insertion portion 12 and holds an ultrasound transducer 15 that acquires information on the subject, and a tubular portion 12b having a tubular shape whose distal end is fitted to a proximal end side of the distal end constituting portion 12a and whose proximal end is connected to the grip portion 13 (see FIG. 4). In addition, a communication hole 12c communicating with a first channel 19 to be described later, a mounting portion 12d having a hole shape and used for mounting the ultrasound transducer 15, and two holding holes 12e connected to the mounting portion 12d and capable of holding insulating pipes 12f in which a part of the signal lines of the signal cable 17 is insertable are provided in the distal end constituting portion 12a. The insulating pipes 12f are made with use of an insulating material, and have cylindrical shapes. The insulating pipes 12f may be obtained by performing an insulation process or the like on a surface of a cylindrical conductive material. Further, the holding holes 12e and holes formed by the insulating pipes 12f constitute communication portions, which allow the mounting portion 12d to communicate with outside, in the distal end constituting portion 12a.

Furthermore, the ultrasound transducer 15, which is an image sensor for acquiring information on the subject, is provided at the distal end of the first insertion portion 12. The ultrasound transducer 15 is configured using, for example, a convex array ultrasound transducer, and a distal end portion of the signal cable 17 is connected thereto. The ultrasound transducer 15 includes a plurality of piezoelectric elements that are arrayed along an axial core of the first insertion portion 12 and arranged so as to perform a fan-like scan on an extension of a central axis of the first insertion portion 12. The ultrasound transducer 15 uses the piezoelectric elements provided at a distal end portion thereof to convert electrical pulse signals received from a control device, such as a signal processing unit to be described later, into ultrasound pulses (acoustic pulses), apply the ultrasound pulses to the subject, convert ultrasound echoes reflected by the subject into electrical echo signals, and outputs the electrical echo signals.

The ultrasound transducer 15 may be any of a convex transducer or a linear transducer. In the first embodiment, it is assumed that the ultrasound transducer 15 is a convex ultrasound transducer that includes a plurality of piezoelectric elements arranged in an array, and electronically switches between the piezoelectric elements used for transmission and reception to thereby electronically perform scan.

Although not illustrated in the drawings, a connector is provided at a proximal end of the universal cord 14, and the connector is connected to the signal processing unit. The signal processing unit transmits a driving signal to the ultrasound transducer 15 via the signal cable 17, processes an ultrasound signal received by the ultrasound transducer 15, generates an ultrasound tomographic image, and displays the ultrasound tomographic image on a monitor (not illustrated).

Further, a water supply port 16 with a cock is provided in an upper part of the grip portion 13. The water supply port 16 communicates with the first channel 19 to be described later, and is able to freely supply perfusate via a perfusion tube (not illustrated). An operator is able to appropriately supply the perfusate into the first channel 19 by opening the cock of the water supply port 16.

The first channel 19 is provided inside the first insertion portion 12 so as to be inclined with respect to an axial direction of the first insertion portion 12. Specifically, a distal end portion of the first channel 19 is opened at a distal end surface of the first insertion portion 12 on the side opposite to the grip portion 13 side, and a proximal end portion of the first channel 19 is opened at a proximal end surface of the first insertion portion 12 on the grip portion 13 side. The proximal end portion of the first channel 19 is positioned on the water supply port 16 side in a radial direction of the first insertion portion 12, and the distal end portion of the first channel 19 is positioned on the side opposite to the the water supply port 16 side in the radial direction of the first insertion portion 12. In the distal end constituting portion 12a, the ultrasound transducer 15 is positioned on the water supply port 16 side, and the opening of the first channel 19 is positioned on the side opposite to the water supply port 16 side when viewed in a longitudinal direction.

Further, the grip portion 13 is provided with an insertion guide hole 13a, a distal end of which communicates with the first channel 19 and a proximal end of which is opened at a proximal end surface of the grip portion 13. In this example, a positioning hole 13b is drilled in the proximal end surface of the grip portion 13, and positioning pins protruding from the optical viewing tube 21 to be described later and the treatment tool guide 22 are fitted into the positioning hole 13b. It may be possible to retain the positioning pins using a fixing screw that fixes the positioning pins to the grip portion 13.

Further, a second insertion portion 21a provided in the optical viewing tube 21 and a third insertion portion 22a provided in the treatment tool guide 22 are selectively inserted in and removed from the first channel 19 of the rigid endoscope body 11. Both of the insertion portions 21a and 22a are rigid and extend linearly. An inner diameter of the first channel 19 is set to a certain size that fits an outer diameter of the second insertion portion 21a. In contrast, an outer diameter of the third insertion portion 22a is set to be approximately equal to the outer diameter of the second insertion portion 21a. Further, a small gap is ensured between an inner periphery of the first channel 19 and an outer periphery of each of the insertion portions 21a and 22a so as to allow the perfusate to circulate. Therefore, the inner diameter of the first channel 19 is set to be slightly greater than the outer diameter of both of the insertion portions 21a and 22a by the gap that allows the perfusate to circulate.

Further, as illustrated in FIG. 1, an eyepiece portion 21b is provided on a front side of the second insertion portion 21a provided in the optical viewing tube 21, and a mouthpiece portion 21c into which a light guide (not illustrated) is inserted is provided in an upper part in the vicinity of a distal end of the eyepiece portion 21b. The light guide passes through the inside of the second insertion portion 21a and extends in a distal end direction, and illumination light transmitted through the light guide is emitted from an illumination window (not illustrated) provided on the distal end portion of the second insertion portion 21a, so that a body cavity wall of the subject is illuminated. Furthermore, an observation window 21d is provided on the distal end of the second insertion portion 21a so as to be adjacent to the illumination window. Reflecting light from the body cavity wall of the subject enters the observation window 21d, and a subject image formed on an optical member, such as an objective lens, provided inside the observation window 21d is transmitted to the eyepiece portion 21b through a relay optical system and then observed.

Moreover, a flange portion 21g is formed on the distal end of the eyepiece portion 21b. A support portion 21e protrudes from a center of a distal end surface of the flange portion 21g. Furthermore, a proximal end portion of the second insertion portion 21a is supported by the support portion 21e. The distal end surface of the flange portion 21g faces the proximal end surface of the grip portion 13 when the second insertion portion 21a is inserted in the rigid endoscope body 11 via the insertion guide hole 13a. In this case, the support portion 21e is inserted through the insertion guide hole 13a. Furthermore, a positioning pin 21f protrudes from a lower part of the distal end surface of the flange portion 21g. The positioning pin 21f is fitted in the positioning hole 13b having an opening at the proximal end surface of the grip portion 13, so that movement in a rotation direction is restricted.

The treatment tool guide 22 includes the third insertion portion 22a, an inducing portion 22b, a flange portion 22c, and a support portion 22d. The inducing portion 22b is provided on a front side of the third insertion portion 22a, and has a funnel shape. Further, the flange portion 22c is provided on the distal end of the inducing portion 22b, the support portion 22d protrudes in the center of the distal end surface, and the proximal end of the third insertion portion 22a is supported by the support portion 22d. The distal end surface of the flange portion 22c faces the proximal end surface of the grip portion 13 when the third insertion portion 22a is inserted in the rigid endoscope body 11 via the insertion guide hole 13a. In this case, the support portion 22d is inserted in the insertion guide hole 13a. Furthermore, a positioning pin 22f protrudes in the lower part of the distal end surface of the flange portion 22c. The positioning pin 22f is fitted in the positioning hole 13b having an opening at the proximal end surface of the grip portion 13, and movement of the positioning pin 22f in a rotation direction is restricted.

A second channel 22e, distal end of which has an opening at the distal end surface of the third insertion portion 22a and a proximal end of which communicates with an induction hole formed in the inducing portion 22b, is provided inside the third insertion portion 22a. An elongated and rigid treatment tool 23b, which linearly extends forward form a device main body 23a and is provided in the treatment tool device 23, can be inserted in and removed from the second channel 22e.

The second channel 22e functions as a guide for inserting and removing the treatment tool 23b, and an inner diameter of the second channel 22e is set to be slightly greater than an outer diameter of the treatment tool 23b. In the first embodiment, the third insertion portion 22a is formed using a pipe material, the inside of the third insertion portion 22a is filled with a resin material, and the second channel 22e is formed in the filling resin material. The second channel 22e may be formed by forming a hole in the third insertion portion 22a made of a sold metallic material.

In the first embodiment, a biopsy device is illustrated as one example of the treatment tool device 23, and a needle portion of the biopsy device corresponds to the treatment tool 23b. Therefore, in the following description, the treatment tool device 23 is replaced with the biopsy device 23, and the treatment tool 23b is replaced with the needle portion 23b.

The needle portion 23b includes a guide tube needle 23c, which has a smaller outer diameter than the second insertion portion 21a of the optical viewing tube 21 and a biopsy needle 23d. The biopsy needle 23d is inserted through the guide tube needle 23c so as to freely move forward and backward. Further, a pocket is formed on a distal end side of the biopsy needle 23d. When a launch button 23e provided on the back surface of the device main body 23a is pressed, the biopsy needle 23d protrudes forward by receiving a resilient force of a spring built in the device main body 23a. Accordingly, the biopsy needle 23d is punctured into tissue of the subject and biopsy tissue is taken into the pocket. When the launch button 23e is pressed, the guide tube needle 23c protrudes following the biopsy needle 23d, and the biopsy tissue is cut out and taken into the pocket when a distal end of the guide tube needle 23c passes over the pocket.

The first channel 19 is arranged at a position protruding toward a scanning surface (observation visual field) of the ultrasound transducer 15. Therefore, when the needle portion 23b is configured to protrude forward from the first channel 19, the needle portion 23b passes through the scanning surface of the ultrasound transducer 15, and accordingly, it becomes possible to display the needle portion 23b in the ultrasonic tomographic image on the monitor.

The needle portion 23b of the present embodiment is inserted through the first channel 19 via the third insertion portion 22a provided in the treatment tool guide 22. Therefore, when the outer diameter of the third insertion portion 22a is set in accordance with the inner diameter of the first channel 19, and the inner diameter of the second channel 22e provided in the third insertion portion 22a is set in accordance with the outer diameter of the needle portion 23b, it is possible to accurately cause the needle portion 23b, which is narrower than the second insertion portion 21a of the optical viewing tube 21, to protrude on the scanning surface of the ultrasound transducer 15.

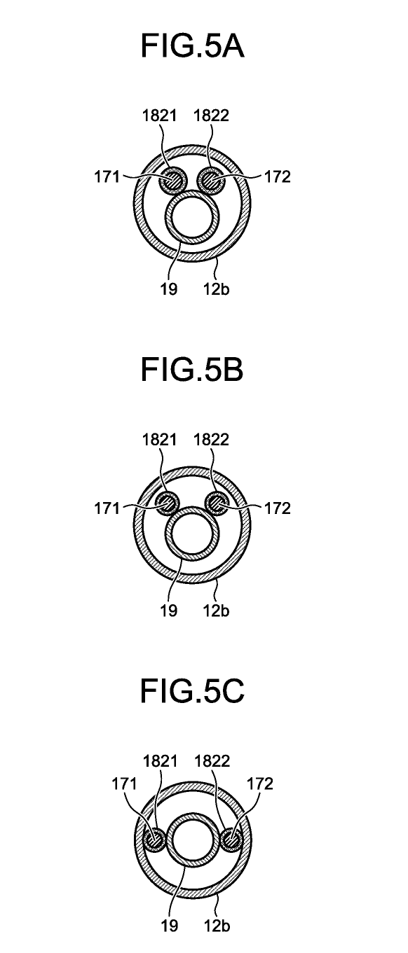

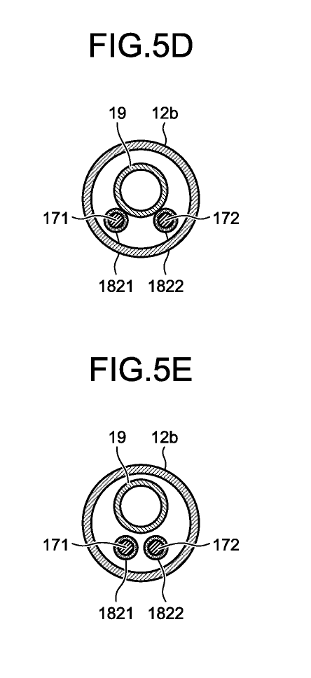

Next, an internal configuration of the rigid endoscope body 11 will be described with reference to FIGS. 3, 4 and 5A to 5E. FIG. 5A is a cross-sectional view of the rigid endoscope body corresponding to line A-A illustrated in FIG. 4. FIG. 5B is a cross-sectional view of the rigid endoscope body corresponding to line B-B illustrated in FIG. 4. FIG. 5C is a cross-sectional view of the rigid endoscope body corresponding to line C-C illustrated in FIG. 4. FIG. 5D is a cross-sectional view of the rigid endoscope body corresponding to line D-D illustrated in FIG. 4. FIG. 5E is a cross-sectional view of the rigid endoscope body corresponding to line E-E illustrated in FIG. 4.

As illustrated in FIG. 3, the signal cable 17 includes: a first cable portion 17a that includes two signal line bundles, one of which is connected to one surface of a relay board 15a and the other one of which is connected to the other surface of the relay board 15a; a binding portion 17b that is connected to the first cable portion 17a and binds the two signal line bundles into a single bundle; and a second cable portion 17c that maintains the single bundle state and extends from the binding portion 17b to the grip portion 13 side. The relay board 15a has a plate shape, and is electrically connected to each of the ultrasound transducer 15 and the signal cable 17.

The first cable portion 17a includes a first signal line group 171 connected to the one surface of the relay board 15a, and a second signal line group 172 connected to the other surface of the relay board 15a. The first signal line group 171 and the second signal line group 172 extend outward from the distal end constituting portion 12a via the two insulating pipes 12f provided on the two holding holes 12e formed in the distal end constituting portion 12a.

The binding portion 17b binds the first signal line group 171 and the second signal line group 172 into a bundle, so that a single bundle of a third signal line group 173 is formed.

In the second cable portion 17c, a comprehensive shield 174 is provided in a part of an outer periphery of the third signal line group 173 (a bundle of a plurality of signal lines), and a jacket 175 is provided in a part of an outer periphery of the comprehensive shield 174. An end portion of the second cable portion 17c on the side opposite to the binding portion 17b is connected to a connector 20 via the grip portion 13.

Further, the signal cable 17 is provided with a first tube 181, a second tube 182, and a third tube 183 (see FIG. 3). Each of the first tube 181, the second tube 182, and the third tube 183 is formed using a heat-shrinkable tube. The first tube 181, the second tube 182, and the third tube 183 cover entire outer peripheries of the signal line groups between the insulating pipe 12f and the comprehensive shield 174 by causing heat shrinkage of the heat-shrinkable tubes to occur while including overlapping regions between at least parts of adjacent tubes.

The first tube 181 covers the two insulating pipes 12f, which are for inserting the first signal line groups 171 and the second signal line group 172, and a part of the first cable portion 17a. The first tube 181 is formed of a first cylindrical portion 1811, which extends along the first signal line group 171 and covers a part of the first signal line group 171, and a second cylindrical portion 1812, which extends along the second signal line group 172 and covers a part of the second signal line group 172.

The second tube 182 covers the first signal line group 171 and the second signal line group 172. One end of the second tube 182 is covered by the first tube 181, and the other end is covered by the third tube 183. The second tube 182 is formed of a first cylindrical portion 1821, which extends along the first signal line group 171 and covers a part of the first signal line group 171, and a second cylindrical portion 1822, which extends along the second signal line group 172 and covers a part of the second signal line group 172.

The third tube 183 covers end portions of the first signal line group 171 and the second signal line group 172 on a side different from a side connected to the relay board 15a, the third signal line group 173 on a side connected to the first cable portion 17a, a part of the comprehensive shield 174, and a part of the jacket 175.

As described above, the first channel 19 is provided so as to be inclined with respect to the axial direction of the first insertion portion 12. Therefore, if the signal cable 17 is provided so as to extend parallel to the central axis of the first insertion portion 12, the signal cable 17 interferes with the first channel 19. In view of the above, in the first embodiment, the first channel 19 is inserted into a space that is formed by dividing the plurality of signal lines into two bundles in the first cable portion 17a, to thereby prevent interference between the signal cable 17 and the first channel 19 (see FIG. 4).

Specifically, a set of the first signal line group 171 and the second signal line group 172 and the first channel 19 are arranged side by side in a vertical direction from the ultrasound transducer 15 side of the first insertion portion 12 in the drawing (see FIG. 5A). At this position, the first cable portion 17a is arranged on the ultrasound transducer 15 side, and the first channel 19 is arranged on the opposite side.

As approaching the grip portion 13 side from the arrangement of FIG. 5A, the first signal line group 171 and the second signal line group 172 are moved in directions opposite to each other along an outer periphery of the first channel 19 (see FIG. 5B to FIG. 5E). At this time, the first channel 19 gradually moves in an upward direction in the drawing along the slope. The arrangement of the signal cable 17 and the first channel 19 is opposite to the arrangement illustrated in FIG. 5A in front of the binding portion 17b. Thereafter, the first signal line group 171 and the second signal line group 172 are collected together by the binding portion 17b. In this manner, it is possible to insert the signal cable 17 and the first channel 19 into the tubular portion 12b by dividing the signal lines of the signal cable 17 into two bundles, without increasing a diameter of the tubular portion 12b and while preventing interference between the signal cable 17 and the first channel 19.

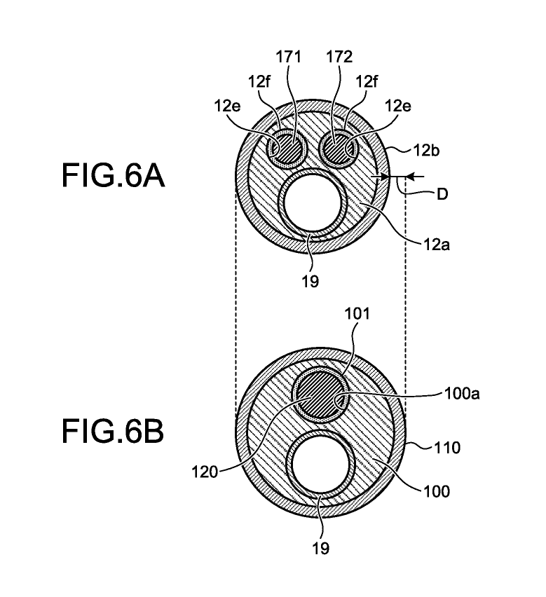

FIGS. 6A and 6B are diagrams for explaining a diameter of the insertion portion according to the first embodiment of the present disclosure and a diameter of a conventional insertion portion. FIG. 6A illustrates a cross section of the distal end constituting portion 12a according to the first embodiment, in particular, a cross section cut along a cutting plane that passes through the insulating pipe 12f. FIG. 6B illustrates a cross section of a conventional distal end constituting portion 100 that has a single holding hole for holding an insulating pipe 101 in which a single signal line group 120 is insertable, in particular, a cross section cut along a cutting plane that passes through the insulating pipe 101. In FIG. 6B, a tubular portion 110 is mounted on the distal end constituting portion 100.

As illustrated in FIGS. 6A and 6B, an outer diameter of the distal end constituting portion 12a, which includes the two holding holes 12e capable of holding the insulating pipes 12f, is smaller than an outer diameter of the distal end constituting portion 100, which includes a single holding hole 100a for holding the insulating pipe 101, by a length D when the minimum thicknesses are set to be the same. In view of the above, it is possible to reduce an outer diameter of the first insertion portion 12, in particular, the outer diameter of the distal end constituting portion 12a, when adopting a configuration in which a plurality of holding holes are provided and the insulating pipes 12f are mounted in the respective holding holes to allow signal lines to extend.

Subsequently, to manufacture the first insertion portion 12 in a process of manufacturing the rigid endoscope body 11 as described above, one end side of a plurality of signal lines, for which the comprehensive shield 174 and the jacket 175 are provided on the one end side, are branched into two, and the third tube 183 before heat shrinkage is inserted from the other end side to the jacket 175.

Thereafter, the above-described second tube 182 before heat shrinkage is inserted. Specifically, the first signal line group 171 is inserted into the first cylindrical portion 1821, and the second signal line group 172 is inserted into the second cylindrical portion 1822. After the signal line is inserted into the second tube 182, the first signal line group 171 and the second signal line group 172 are inserted into the first tube 181 before heat shrinkage and the distal end constituting portion 12a in this order. At this time, the insulating pipes 12f are fitted into the holding holes 12e of the distal end constituting portion 12a.

Thereafter, the first cable portion 17a and the relay board 15a are connected to each other. In this case, the ultrasound transducer 15 may be connected to the relay board 15a in advance, or the ultrasound transducer 15 may be connected to the relay board 15a after connecting the first signal line group 171 and the second signal line group 172 to the relay board 15a. After the first cable portion 17a and the relay board 15a are connected, the ultrasound transducer 15 is housed in the distal end constituting portion 12a, and the ultrasound transducer 15 is fixed to the distal end constituting portion 12a by bonding.

Thereafter, positions of the first tube 181 and the third tube 183 before heat shrinkage and the signal lines are adjusted such that the first tube 181 and the third tube 183 cover parts of the second tube 182, and the first tube 181, the second tube 182, and the third tube 183 are heated to cause heat shrinkage to occur so as to be crimped to the signal lines. It is preferable that a length of the overlapping portion where each of the first tube 181 and the third tube 183 covers a part of the second tube 182 is set to be equal to or greater than 4 millimeters (mm).

Thereafter, the first channel 19 is inserted into the space formed by the first signal line group 171 and the second signal line group 172. Thereafter, the signal cable 17 and the first channel 19 are inserted into the tubular portion 12b, and the tubular portion 12b is mounted on the distal end constituting portion 12a, so that the first insertion portion 12, into which the signal cable 17 and the first channel 19 are inserted, is formed.

According to the first embodiment as described above, the two bundles of signal line groups (the first signal line group 171 and the second signal line group 172) extend from the distal end constituting portion 12a via the two insulating pipes 12f provided in the distal end constituting portion 12a, and the first channel 19 is inserted into the space formed by the two signal line groups. Therefore, it is possible to prevent disconnection of the signal lines due to load applied in the vicinity of the distal end constituting portion 12a and reduce the diameter of the first insertion portion 12.

Furthermore, according to the first embodiment as described above, the binding portion 17b binds end portions of the signal cable 17 on a side opposite to the distal end constituting portion 12a side into a single bundle. Therefore, as compared to the two-bundle state, it is possible to improve performance of operation of inserting the signal cable 17 into the tubular portion 12b at the time of manufacturing an endoscope.

Moreover, according to the first embodiment as described above, the signal line groups exposed between the distal end constituting portion 12a and the comprehensive shield 174 are covered by the heat-shrinkable tubes (the first tube 181, the second tube 182, and the third tube 183) having insulation properties. Therefore, it is possible to ensure insulation properties of the signal line groups. In particular, a branch portion (the binding portion 17b) of the signal line groups is covered by the second tube 182 and the third tube 183 overlapping with each other, so that it is possible to reliably ensure an insulation property at the branch portion.

In the first embodiment as described above, a case has been described in which the positions of the first tube 181 and the third tube 183 are adjusted, and the first tube 181, the second tube 182, and the third tube 183 before heat shrinkage are heated to cause heat shrinkage to occur to thereby cover the plurality of signal lines. However, it may be possible to heat a region in which the tubes overlap with each other, such as a region in which the first tube 181 and the second tube 182 overlap with each other and a region in which the second tube 182 and the third tube 183 overlap with each other, to cause heat shrinkage to occur in only the region in which the tubes overlap with each other, to thereby cause parts of the tubes to be firmly attached to each other.

Furthermore, in the first embodiment as described above, markers indicating arrangement positions of the first tube 181 and the third tube 183 with respect to the second tube 182 may be provided on the second tube 182 (the second tube before heat shrinkage). With this configuration, it is possible to arrange the first tube 181 and the third tube 183 before heat shrinkage while checking the positions with respect to the second tube before heat shrinkage.

### Second Embodiment

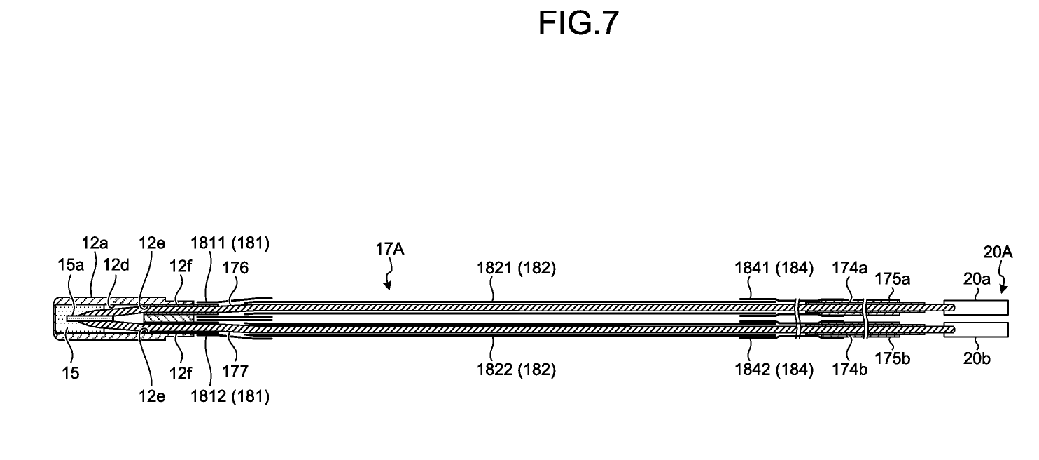

In the first embodiment as described above, a case has been described in which the two bundles of signal line groups are bound into a single bundle in the signal cable 17. However, it may be possible to connect the two bundles as they are to the connector. FIG. 7 is a cross-sectional view schematically illustrating a configuration of a main part of a rigid endoscope body of a rigid endoscope system according to a second embodiment of the present disclosure.

As illustrated in FIG. 7, a signal cable 17A according to the second embodiment includes a first signal line group 176 connected to one surface of the relay board 15a, and a second signal line group 177 connected to the other surface of the relay board 15a. The first signal line group 176 and the second signal line group 177 extend outward from the distal end constituting portion 12a via the two insulating pipes 12f provided in the two holding holes 12e formed in the distal end constituting portion 12a, and end portions thereof on the side opposite to the distal end constituting portion 12a side are respectively connected to a first connector 20a and a second connector 20b of a connector 20A. A first comprehensive shield 174a is provided in a part of an outer periphery of the first signal line group 176, and a first jacket 175a is provided on an outer periphery of the first comprehensive shield 174a. Further, a second comprehensive shield 174b is provided in a part of an outer periphery of the second signal line group 177, and a second jacket 175b is provided on an outer periphery of the second comprehensive shield 174b.

Furthermore, the signal cable 17A is provided with the first tube 181, the second tube 182, and a third tube 184 as described above. The first cylindrical portion 1821 of the second tube 182 is provided between the distal end constituting portion 12a and the first comprehensive shield 174a in the first signal line group 176. In contrast, the second cylindrical portion 1822 is provided between the distal end constituting portion 12a and the second comprehensive shield 174b in the second signal line group 177. The second tube 182 is formed using a heat-shrinkable tube, and covers parts of a plurality of signal lines including regions overlapping with the first tube 181 and the third tube 184 at both ends thereof.

The third tube 184 is formed of a first cylindrical portion 1841, which extends along the first signal line group 176, and a second cylindrical portion 1842, which extends along the second signal line group 177. The first cylindrical portion 1841 covers an end portion of the first cylindrical portion 1821, a part of the first signal line group 176, and an end portion of the first comprehensive shield 174a. The second cylindrical portion 1842 covers an end portion of the second cylindrical portion 1822, a part of the second signal line group 177, and an end portion of the second comprehensive shield 174b.

Even in the second embodiment, similarly to the first embodiment as described above, the first channel 19 is inserted into a space that is formed by dividing the plurality of signal lines into two bundles in the signal cable 17A, to thereby prevent interference between the signal cable 17A and the first channel 19 (for example, see FIG. 4).

According to the second embodiment as described above, the two bundles of signal line groups (the first signal line group 176 and the second signal line group 177) extend from the distal end constituting portion 12a via the two insulating pipes 12f provided in the distal end constituting portion 12a, and the first channel 19 is inserted into the space formed by the two signal line groups. Therefore, it is possible to prevent disconnection of the signal lines due to load applied in the vicinity of the distal end constituting portion 12a and reduce the diameter of the first insertion portion 12.

Furthermore, according to the second embodiment, the two bundles of signal line groups (the first signal line group 176 and the second signal line group 177) are extended as they are and connected to the connector 20A. Therefore, a branch portion of the signal lines is not present between the second tube 182 and the third tube 184, so that is possible to easily arrange the heat-shrinkable tubes as compared to the first embodiment as described above.

While the embodiments of the present disclosure have been described above, the disclosure is not limited to only the above-described embodiments and modifications. The disclosure is not limited to the embodiments and modifications as described above, and various embodiments may be made within the scope not departing from the technical concept as defined by the appended claims. In addition, configurations of the embodiments and modifications may be combined appropriately.

Furthermore, according to the first and second embodiments as described above, the piezoelectric element has been described as one example of a device that outputs an ultrasound wave and converts an ultrasound wave entered from outside into an echo signal; however, the embodiments are not limited to this example. It may be possible to adopt a device manufactured using microelectromechanical systems (MEMS), such as capacitive micromachined ultrasonic transducers (C-MUTs).

Moreover, according to the first and second embodiments as described above, the ultrasound endoscope that observes the inside of the subject via the urethra has been described. However, a device that is inserted into a biliary tract, a bile duct, a pancreatic duct, a trachea, a bronchus, or a ureter other than the urethra and observes surrounding organs (a pancreas, lungs, a bladder, lymph nodes, and the like).

Furthermore, according to the first and second embodiments as described above, the ultrasound endoscope has been described as one example; however, the embodiments are not limited to this example as long as the endoscope includes a signal cable for transmitting an image signal. For example, the disclosure is applicable to an oral endoscope that is inserted into a digestive tract (an esophagus, a stomach, a duodenum, or a large intestine) or a respiratory organ (a trachea or a bronchus) of the subject and captures an image of digestive tracts and respiratory organs, that is, the oral endoscope provided with a flexible insertion portion that includes an imaging element serving as an image sensor. In particular, the disclosure is useful for an endoscope provided with an image sensor that includes a cable having a large number of signal lines and requiring insulation process, such as a charge coupled device (CCD) used for a high-speed camera. If the image sensor is an imaging element and it is not necessary to ensure an insulation property, it may be possible not to provide an insulating pipe but extend a signal line group (the first signal line group 171 or the second signal line group 172) from the distal end constituting portion 12a via the holding hole 12e. In this case, the holding hole 12e serves as the communication portion.

Moreover, according to the first and second embodiments as described above, a case has been described in which the two holding holes 12e are formed in the distal end constituting portion 12a and each of the holding holes 12e holds the insulating pipe 12f. However, it may be possible to form a single holding hole capable of collectively holding the two insulating pipes 12f. In this case, each of the two insulating pipes 12f held by the holding hole serves as the communication portion.

According to some embodiments, it is possible to prevent disconnection and reduce a diameter.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an insertion portion configured to be inserted into a subject;
an image sensor configured to acquire an image of the subject;
a distal end constituting portion that is provided at a distal end of the insertion portion, and that includes a mounting portion having a hole shape and used for mounting the image sensor, and two communication portions for allowing the mounting portion to communicate with outside of the distal end constituting portion;
a signal cable that includes two signal line groups, the signal line groups having one ends connected to the image sensor, extending from the distal end constituting portion via the communication portions, and including a plurality of signal lines configured to transmit signals acquired by the image sensor;
a channel that has a cylindrical shape, that is provided inside the insertion portion, that is inserted into a space formed by the two signal line groups of the signal cable, and that allows an elongated member to be inserted in the channel; and
a tubular portion that has a tubular shape and allows the signal cable and the channel to be inserted in the tubular portion.

2. The endoscope according to claim 1, further comprising:
an insulating first tube provided on the two signal line groups on a side of the distal end constituting portion, the insulating first tube being configured to cover each of the signal line groups; and
an insulating second tube that has one end overlapping with the first tube, and that extends toward a side opposite to the side of the distal end constituting portion along a longitudinal direction of the signal line groups, the insulating second tube being configured to cover each of the signal line groups.

3. The endoscope according to claim 2, wherein a part of the first tube is configured to cover the second tube and is firmly attached to a part of the second tube.

4. The endoscope according to claim 1, wherein the image sensor is an ultrasound transducer, and the communication portions include insulating pipes having insulation properties.

5. The endoscope according to claim 1, wherein the signal cable includes a binding portion configured to bind the two signal line groups into a single bundle.

6. The endoscope according to claim 1, wherein the two signal line groups extend over an entire length of the signal cable.

7. An endoscope comprising:
a tubular portion that has a tubular shape;
a channel that is inserted into the tubular portion and arranged to be inclined with respect to an axial direction of the tubular portion;
a signal cable including two signal line groups by which a space is formed, the space allowing the channel to be inserted in the space;
an ultrasound transducer connected to a distal end of the signal cable and configured to acquire information on a subject;
a distal end constituting portion that is provided at a distal end of the tubular portion, and that includes a mounting portion on which the ultrasound transducer is mounted, a holding hole capable of holding an insulating pipe in which the signal cable is insertable, and a communication hole communicating with the channel; and
an insulating tube configured to cover a part of each of the two signal line groups of the signal cable.
